(19) United States
(12) Patent Application Publication
(10) Pub. No.: US 2018/0155261 A1
(43) Pub. Date: Jun. 7, 2018

(54) METHOD FOR CONVERTING NATURAL GAS TO DIMETHYL ETHER

(71) Applicant: ExxonMobil Research and Engineering Company, Annandale, NJ (US)

(72) Inventor: Michael F. Raterman, Doylestown, PA (US)

(21) Appl. No.: 15/805,265

(22) Filed: Nov. 7, 2017

Related U.S. Application Data
(60) Provisional application No. 62/423,327, filed on Nov. 17, 2016.

(51) Int. Cl. C07C 27/06; C07C 41/09; C07C 43/04; C07C 29/151; C07C 31/04; B01D 5/00; B01D 19/00

(52) U.S. Cl. CPC C07C 27/06; C07C 41/09; C07C 43/043; C07C 29/1516; C01B 2203/1241; B01D 5/009; B01D 19/0068; C01B 2203/025; C07C 31/04

(57) ABSTRACT

Systems and methods for upgrading natural gas that may include the division of a natural gas feed steam into two parts—one that is partially oxidized into syngas and a second that is “dry” reformed into syngas with the assistance of heat from the partial oxidation. Each of the resulting syngas products may then be combined, and after water is condensed from the syngas, the combined syngas product may be converted to dimethyl ether.

### CROSS-REFERENCE TO RELATED APPLICATIONS

This application claims the benefit of U.S. Provisional Application No. 62/423,327, filed on Nov. 17, 2016, the entire contents of which are incorporated herein by reference.

### FIELD

The present application relates to a method for converting natural gas to dimethyl ether.

### BACKGROUND

With the development of shale gas resources, there is an opportunity to monetize these assets. While liquefying natural gas to make LNG or CNG product may be a good option for larger production sites, the economics are less favorable for smaller and geographically dispersed natural gas production sites.

The present application relates to methods and systems that may be employed at such production sites, or elsewhere, to convert natural gas to dimethyl ether.

### SUMMARY

In one aspect, a method is provided for upgrading natural gas. The method includes partially oxidizing a first natural gas feed stream to make a first syngas product; heating a second natural gas feed stream with the first syngas product; reforming the second natural gas feed stream and carbon dioxide to make a second syngas product; combining the first syngas product and second syngas product into a combined syngas product; and converting the combined syngas product to a converted product stream comprising dimethyl ether.

In another aspect, a system is provided for upgrading natural gas. The system includes a first natural gas feed stream; a second natural gas feed stream; a partial oxidation unit receiving the first natural gas feed stream, the partial oxidation unit operating under conditions to partially oxidize the first natural gas stream to make a first syngas product; a reformer receiving the second natural gas stream and carbon dioxide, the reformer operating under conditions to react the second natural gas stream and carbon dioxide to make a second syngas product; and a reaction unit receiving the first syngas product and second syngas product and produce a converted product comprising dimethyl ether.

### DRAWINGS

FIG. 1 is a schematic illustrating a method and system for converting natural gas to dimethyl ether according to one or more embodiments of the present invention.

### DETAILED DESCRIPTION

Disclosed herein are systems and methods for upgrading natural gas that may include the division of a natural gas feed steam into two parts—one that is partially oxidized into syngas (e.g., converted into syngas in accordance with the following Eq. 1) and a second that is “dry” reformed into syngas with the assistance of heat from the partial oxidation in accordance with the following Eq. 2.

2CH4+O2⇔2CO+4H2 (Eq. 1)

CH4+CO2⇔2CO+2H2 (Eq. 2)

Each of the resulting syngas products may then be combined, and after water is condensed from the syngas, the combined syngas product may be converted to dimethyl ether. Advantageously, the dimethyl ether may be used as a solvent to carry the CO2 in the product until it is separated from the dimethyl ether and used as a recycle to feed to the dry reforming stage of the process. Such systems may be advantageously employed at natural gas production sites, and may be particularly beneficial at sites that produce 50 million SCF per day or less. In addition, in some embodiments, such systems may advantageously operate with compressed ambient air as an oxidizing feed without the requirement of an air separation unit or an available enriched oxygen supply (e.g., from an oxygen pipeline).

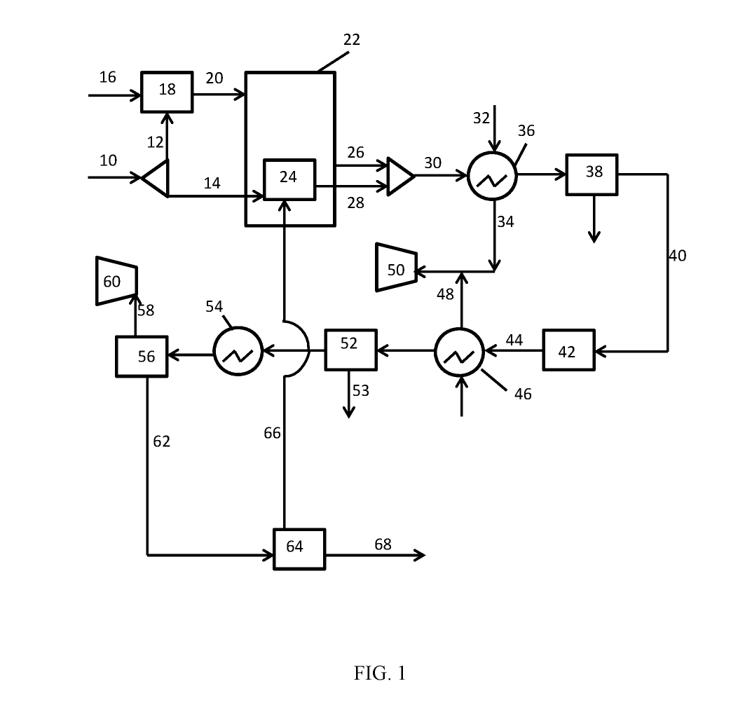

As illustrated in FIG. 1, a natural gas feed stream 10, which has preferably already had H2S removed from the stream is split into natural gas feed stream 12 and natural gas feed stream 14. Stream 12 is fed with compressed air 16 to a partial oxidation unit 18 where the natural gas is converted into a syngas product, e.g., in accordance with Eq. 1. Compressed air 14 may be compressed ambient air, or it may be an enriched oxygen supply, such as an enriched oxygen feed from an oxygen pipeline or an enriched oxygen feed from an air separation unit. The partial oxidation unit 18 operates under conditions suitable for converting the natural gas to syngas, for example, at a temperature of 1400° F. to 2700° F., such as 1900° F. to 2500° F., or 2000° F. to 2400° F. or about 2200° F., and a pressure of about 200 to 1200 psia, such as bout 500 to 1000 psia, or 600 to 700 psia or about 665 psia. The reaction in partial oxidation unit 18 may be performed under dry conditions, e.g., without the addition of water upstream of the partial oxidation unit 18.

The syngas product 20 is fed to a reforming unit 22 where it used to preheat one or more feeds to a reforming reactor 24 and/or heat the reactor vessel, itself. The reforming reactor 24 receives the natural gas feed stream 14 and a carbon dioxide feed 66 and converts the natural gas to syngas in accordance with Eq. 2. The carbon dioxide feed 66 may be a recycle feed, as shown, and/or it may include carbon dioxide from another source, e.g., field carbon dioxide. In an exemplary embodiment, the reforming unit 22 may include a reforming reactor 24 that is in the form of a tubular reactor in which hot gas from the partial oxidation unit 18 flows around the tubes, thereby exchanging heat with the tubes. The reactor 24 may include a catalyst suitable for converting natural gas and carbon dioxide to syngas. For example, typical catalyst may be noble metals, Ni, NI alloys on alumina either individually or in combination and may contain modifiers such a Cr or Mg. The reaction in reforming reactor 24 may be performed under dry conditions, e.g., without the addition of water upstream of the reforming reactor 24.

The syngas product 28 leaving the reforming reactor 24 may exit the reactor at a temperature of 1400° F. to 2700° F., such as 1900° F. to 2500° F., or 2000° F. to 2400° F. or about 2200° F., and a pressure of about 200 to 1200 psia, such as bout 500 to 1000 psia, or 600 to 700 psia or about 665 psia. The syngas product 28 may comprise about 40 mole % CO and about 40 mole % H2. The syngas product 28 is then combined with syngas product 26 from the partial oxidation unit 18 for conversion to dimethyl ether. The combined syngas product may have a molar H2:CO ratio of between 1.1:1 and 1.9:1, such as between 1.3:1 and 1.7:1.

The combined syngas product 30 is fed to heat exchanger 36 which heats water 32 into steam 34 for energy recovery at a steam turbine generator 50. The combined syngas product 36 is then fed to a separator 38, operating under conditions suitable for condensing water in the combined syngas product 36 and separating the water 39 from the syngas 40. For example, the separator 38 may operate at a temperature of about 210° F. or less, such as about 200° F. or less. After water is condensed out, the syngas 40 may comprise 25 to 40 mole % H2, 40 to 50 mole % N2, 15 to 25 mole % CO, 0 to 5 mole % CO2, and about 1 mole % H2O or less, for example , the syngas may comprise about 33 mole % H2,about 45 mole % N2, about 20 mole % CO, about 2 mole % CO2, and less than 1 mole % H2O.

The syngas 40 may then be reheated to 400° F. to 465° F. before being fed to dimethyl ether reactor unit 42. The dimethyl ether reactor unit 42 may include a reactor operating under conditions suitable for converting syngas to dimethyl ether. Various reaction pathways may be employed to convert syngas to dimethyl ether. In an exemplary embodiment, the syngas may be converted to dimethyl ether via a methanol intermediate. For example, the syngas may first be converted to methanol in accordance with Eq. 3, and then the methanol may be dehydrated to dimethyl ether in accordance with Eq. 4. The dehydration may be performed substantially instantaneously with the synthesis of methanol using a catalyst system that includes a first catalyst for methanol synthesis and a second catalyst for methanol dehydration. These catalysts may be contained on the same, common particle, or the catalysts may be on separately, intermixed particles.

2H2+CO⇔CH3OH (Eq. 3)

2CH3OH⇔CH3OCH3+H2O (Eq. 4)

In an exemplary embodiment, the reactor may be a radial flow reactor. The reactor may be maintained at or near isothermal conditions by heat removal, quenching, or indirect heat exchange. The reactor may include a catalyst system including one or more catalysts. The term “catalyst system” as used herein can refer to one or more catalysts suitable for producing the reaction products from the feeds described. In the context of a dimethyl ether reactor, the catalyst system may include a first catalyst for converting CO and H2 into methanol and a second catalyst for converting methanol to dimethyl ether. The first catalyst may be a M1/M2/Al catalyst, wherein M1 can be Cu, Ag, Au, Ru, Rh, Pd, Re, Os, Ir, Pt, and M2 can be Ti, V, Cr, Mn, Fe, Co, Ni, Zn, rare earth metals La series and Y series. M1 can be single metal, or the combinations of the M1 metal mixtures, M2 can be single metal, or the combinations of the M2 metal mixtures. The second catalyst may be an acid catalyst, such as a zeolite, ion exchanged zeolite e.g. SAPO, alumina, alumina silicates, titania, zirconia, and mixtures of the combinations of the acidic components, e.g., WO3/ZrO2, ZrO2/SiO2, resins, MOFS, ZIFs. The acidic property can be Lewis acidity, or Bronsted acidity, and the combination of the both Lewis acidity and Bronsted acidity. The metal components can be mixed with acidic components, or impregnated onto acidic supports, or extruded with acidic components. By employing the catalyst for conversion of syngas to methanol and the catalyst for the dehydration of methanol to dimethyl ether in the same reactor, the immediate in-situ dehydration of methanol can be achieved, improving the performance of the acid catalyst. The catalyst system may be a physical mixture of the catalysts or it may be a particle combining both catalyst functionalities. Although specific examples are described, the methanol synthesis catalyst may be any catalyst suitable for promoting the conversion of syngas to methanol, and the methanol dehydration catalyst may be any catalyst suitable for dehydrating methanol to dimethyl ether.

The effluent 44 from the reactor unit 42 comprises dimethyl ether, hydrogen, carbon dioxide, carbon monoxide, and water. The effluent44 may also include nitrogen when compressed air is used in the partial oxidation unit 18. In an exemplary embodiment, the effluent 44 may contain about 5 to about 25 mole % dimethyl ether, about 50 to about 75 mole % nitrogen, about 1 to about 15 mole % unreacted hydrogen, about 1 to about 15 mole % unreacted carbon monoxide, about 1 to about 20 mole % carbon dioxide and about 1 to about 20 mole % water. For example, the effluent 44 may contain about 11 % dimethyl ether, about 65 mole % nitrogen, about 6 mole % unreacted hydrogen, about 2 mole % unreacted carbon monoxide, about 6.5 mole % carbon dioxide and about 7 mole % water.

Although not illustrated, the effluent 44 of the reactor unit 42 may be used to preheat the feed to the reactor unit 42. The effluent 44 may then be chilled in heat exchanger 46 with water. The resulting steam 48 may be fed to the steam turbine generator 50. The effluent 44 may then be further chilled to a temperature suitable for condensing water 53 from the effluent 44 when it is fed to separator 52, which may be operated at, for example, about 80° F. and about 650 psia. The effluent 44, once water is condensed, may then be chilled to −80° F. to −85° F. in a chiller 54 to allow for dimethyl ether 62 to be condensed and separated from unreacted hydrogen 58 in a high pressure separator 56. For example, 98 to 99 mole % of the dimethyl ether may be recovered as condensed liquid from the separator 56. Along with the dimethyl ether, a majority of the carbon dioxide (for example, about 66 mole %) in the effluent 44, as well as some of the nitrogen and carbon monoxide are condensed. Most of the nitrogen and most of the hydrogen remains in the vapor phase. The vapor at about 650 psia can be expanded across a turbine to recover power before further energy recovery. Another benefit to using a turbine expander is that the vapor may be further chilled probiding additional refrigeration for condensing dimethyl ether in chiller 54. The hydrogen 58 may be combusted, for example, in a gas turbine generator 60 for further energy recovery.

Advantageously, dimethyl ether is a good solvent for carbon dioxide, and carbon dioxide stream 66 may be recovered from the dimethyl ether stream 62 in dimethyl ether recovery unit 64, leaving dimethyl ether product 68. The dimethyl ether stream 62 may contain abount 50 mole % to about 75 mole % dimethyl ether, about 15 to about 35 mole % carbon dioxide, about 2 to about 10 mole % nitrogen and about 1 to about 10 mole % methanol. The dimethyl ether recovery unit 64 may include any type of separation unit suitable for separating carbon dioxide and dimethyl ether, such as a single or multi-stage fractionation unit (e.g., a unit with overhead conditions of −20° F. and 250 psia), an adsorber, or a stripper. Because carbon dioxide stream 66 is recycled for use as a feed to the reforming reactor 24, the overall carbon efficiency of the process, measured as the number of carbon atoms recovered as dimethyl ether divided by the number of carbon atoms fed to the system as natural gas multiplied by 100%, may be greater than 50%, such as greater than 70%, or greater than 75%.

### Embodiments

The following embodiments are also contemplated:

Embodiment 1—A method of upgrading natural gas comprising: partially oxidizing a first natural gas feed stream to make a first syngas product; heating a second natural gas feed stream with the first syngas product; reforming the second natural gas feed stream and carbon dioxide to make a second syngas product; combining the first syngas product and second syngas product into a combined syngas product; and converting the combined syngas product to a converted product stream comprising dimethyl ether.

Embodiment 2—The method or system of any other enumerated Embodiment, further comprising condensing water from the combined syngas product.

Embodiment 3—The method or system of any other enumerated Embodiment, wherein the combined syngas product comprises less than 1 mole % water after the step of condensing water from the combined syngas product.

Embodiment 4—The method or system of any other enumerated Embodiment, wherein at least 50% of carbon in the first and second natural gas feed streams is converted to dimethyl ether.

Embodiment 5—The method or system of any other enumerated Embodiment, wherein at least 70% of carbon in the first and second natural gas feed streams is converted to dimethyl ether.

Embodiment 6—The method or system of any other enumerated Embodiment, wherein the step of converting the combined syngas product to dimethyl ether comprises contacting the combined syngas product with a catalyst system comprising a first catalyst for converting syngas to methanol an a second catalyst for dehydrating methanol to dimethyl ether.

Embodiment 7—The method or system of any other enumerated Embodiment, wherein the first catalyst and the second catalyst are present in a common catalyst particle.

Embodiment 8—The method or system of any other enumerated Embodiment, wherein the converted product stream further comprises carbon dioxide.

Embodiment 9—The method or system of any other enumerated Embodiment, further comprising separating carbon dioxide from the converted product stream and recycling the carbon dioxide to use in the step of reforming the second natural gas feed stream.

Embodiment 10—The method or system of any other enumerated Embodiment, further comprising separating hydrogen from the converted product stream.

Embodiment 11—The method or system of any other enumerated Embodiment, further comprising combusting the hydrogen separated from the converted product stream in a gas turbine generator.

Embodiment 12—The method or system of any other enumerated Embodiment, wherein air is used to partially oxidize the first natural gas feed stream to make the first syngas product.

Embodiment 13—The method or system of any other enumerated Embodiment, wherein an enriched oxygen stream is used to partially oxidize the first natural gas feed stream to make the first syngas product.

Embodiment 14—The method or system of any other enumerated Embodiment, wherein the first gas feed stream is partially oxidized at a temperature of 1400° F. to 2700° F. and a pressure of 200 to 1200 psia.

Embodiment 15—The method or system of any other enumerated Embodiment, wherein the second syngas product is produced in a reforming reactor having an outlet temperature between 1400° F. and 2700° F. and a pressure of 200 to 1200 psia.

Embodiment 16—The method or system of any other enumerated Embodiment, wherein the step of condensing water comprises cooling the combined syngas product to about 210° F. or less.

Embodiment 17—The method or system of any other enumerated Embodiment, wherein the combined syngas product comprises a molar H2:CO ratio of between 0.9:1 and 2.3:1.

Embodiment 18—The method or system of any other enumerated Embodiment, wherein the molar H2:CO ratio is between 1.3:1 and 1.7:1.

Embodiment 19—The method or system of any other enumerated Embodiment, wherein the dimethyl ether is produced in a dimethyl ether reactor and the combined syngas product fed to the dimethyl ether reactor comprises 25 to 40 mole % H2, 40 to 50 mole % N2, and 15 to 25 mole % CO.

Embodiment 20—The method or system of any other enumerated Embodiment, wherein the combined syngas product fed to the dimethyl ether reactor comprises about 1% or less H2O.

Embodiment 21—The method or system of any other enumerated Embodiment, wherein the step of reforming the second natural gas feed stream and carbon dioxide to make the second syngas product is performed without the addition of water.

Embodiment 22—A system for upgrading natural gas comprising: a first natural gas feed stream; a second natural gas feed stream; a partial oxidation unit receiving the first natural gas feed stream, the partial oxidation unit operating under conditions to partially oxidize the first natural gas stream to make a first syngas product; a reformer receiving the second natural gas stream and carbon dioxide, the reformer operating under conditions to react the second natural gas stream and carbon dioxide to make a second syngas product; and a reaction unit receiving the first syngas product and second syngas product and produce a converted product comprising dimethyl ether.

1. A method of upgrading natural gas comprising:
partially oxidizing a first natural gas feed stream to make a first syngas product;
heating a second natural gas feed stream with the first syngas product;
reforming the second natural gas feed stream and carbon dioxide to make a second syngas product;
combining the first syngas product and second syngas product into a combined syngas product; and
converting the combined syngas product to a converted product stream comprising dimethyl ether.

2. The method of claim 1, further comprising condensing water from the combined syngas product.

3. The method of claim 2, wherein the combined syngas product comprises less than 1 mole % water after the step of condensing water from the combined syngas product.

4. The method of claim 1, wherein at least 50% of carbon in the first and second natural gas feed streams is converted to dimethyl ether.

5. The method of claim 1, wherein at least 70% of carbon in the first and second natural gas feed streams is converted to dimethyl ether.

6. The method of claim 1, wherein the step of converting the combined syngas product to dimethyl ether comprises contacting the combined syngas product with a catalyst system comprising a first catalyst for converting syngas to methanol an a second catalyst for dehydrating methanol to dimethyl ether.

7. The method of claim 6, wherein the first catalyst and the second catalyst are present in a common catalyst particle.

8. The method of claim 1, wherein the converted product stream further comprises carbon dioxide.

9. The method of claim 8, further comprising separating carbon dioxide from the converted product stream and recycling the carbon dioxide to use in the step of reforming the second natural gas feed stream.

10. The method of claim 1, further comprising separating hydrogen from the converted product stream.

11. The method of claim 1, further comprising combusting the hydrogen separated from the converted product stream in a gas turbine generator.

12. The method of claim 1, wherein air is used to partially oxidize the first natural gas feed stream to make the first syngas product.

13. The method of claim 1, wherein an enriched oxygen stream is used to partially oxidize the first natural gas feed stream to make the first syngas product.

14. The method of claim 1, wherein the first gas feed stream is partially oxidized at a temperature of 1400° F. to 2700° F. and a pressure of 200 to 1200 psia.

15. The method of claim 1, wherein the second syngas product is produced in a reforming reactor having an outlet temperature between 1400° F. and 2700° F. and a pressure of 200 to 1200 psia.

16. The method of claim 2, wherein the step of condensing water comprises cooling the combined syngas product to about 210° F. or less.

17. The method of claim 1, wherein the combined syngas product comprises a molar H₂:CO ratio of between 0.9:1 and 2.3:1.

18. The method of claim 1, wherein the molar H₂:CO ratio is between 1.3:1 and 1.7:1.

19. The method of claim 1, wherein the dimethyl ether is produced in a dimethyl ether reactor and the combined syngas product fed to the dimethyl ether reactor comprises 25 to 40 mole % H₂, 40 to 50 mole % N₂, and 15 to 25 mole % CO.

20. The method of claim 19, wherein the combined syngas product fed to the dimethyl ether reactor comprises about 1% or less H₂O.

21. The method of claim 1, wherein the step of reforming the second natural gas feed stream and carbon dioxide to make the second syngas product is performed without the addition of water.

22. A system for upgrading natural gas comprising:
a first natural gas feed stream;
a second natural gas feed stream;
a partial oxidation unit receiving the first natural gas feed stream, the partial oxidation unit operating under conditions to partially oxidize the first natural gas stream to make a first syngas product;
a reformer receiving the second natural gas stream and carbon dioxide, the reformer operating under conditions to react the second natural gas stream and carbon dioxide to make a second syngas product; and
a reaction unit receiving the first syngas product and second syngas product and produce a converted product comprising dimethyl ether.
